(19) United States
(12) Patent Application Publication
(10) Pub. No.: US 2011/0112649 A1
(43) Pub. Date: May 12, 2011

(54) Radial and ulnar replacement

(75) Inventors: Bryce Isch, Bluffton, IN (US); Nick Katrana, Fort Wayne, IN (US); Adam Finley, Leesburg, IN (US)

(73) Assignee: Biomet Manufacturing Corp., Warsaw, IN (US)

(21) Appl. No.: 12/590,463

(22) Filed: Nov. 9, 2009

(51) Int. Cl. A61F 2/38

(52) U.S. Cl. 623/20.12

(57) ABSTRACT

Prostheses are provided for ulna and radius of a patient's forearm.

### FIELD OF THE INVENTION

The present invention relates to prostheses for the forearm, namely for the ulna and radius.

### BACKGROUND OF THE INVENTION

The invention relates to prostheses that are used to replace a missing or diseased portion of bone in the forearm. Several conditions can lead to the loss of bone including trauma, arthritic diseases, tumors, musculoskeletal defects, and the replacement of a failed implant. It is desirable to provide prostheses for the ulna and radius that are superior to those revealed prior.

### SUMMARY OF THE INVENTION

To make manifest the above noted and other manifold desires, a revelation of the present invention is brought forth. In a preferred embodiment, the present invention brings forth various replacement and partial replacement ulna and radial prostheses.

Further areas of applicability of the present invention will become apparent from the detailed description provided hereinafter. It should be understood that the detailed description and specific examples, while indicating preferred embodiments of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 is a perspective view of a patient's forearm illustrating positions of the radius and ulna within a patient's forearm;

FIG. 2 is a front elevational view of a replacement ulna prosthesis according to the present invention;

FIG. 3 is a sectional view taken along line 3-3 of FIG. 2;

FIG. 4 is a front elevational view of an alternate preferred embodiment ulna prosthesis according to the present invention;

FIG. 5A is a front elevational view of another alternate preferred embodiment replacement ulnar prosthesis according to the present invention;

FIG. 5B is a front elevational view of a replacement radial prosthesis according to the present invention;

FIG. 6 is a front elevational view of an alternate preferred embodiment replacement radial prosthesis of the present invention;

FIG. 7 is a front elevational view of a partial distal radial prosthesis according to the present invention;

FIG. 8 is a front elevational view of a partial proximal radial prosthesis according to the present invention;

FIG. 9 is a front elevational view of a partial proximal ulnar prosthesis according to the present invention;

FIG. 10 is a front elevational view of a partial distal ulnar prosthesis according to the present invention;

FIG. 11 is a front elevational view of an alternative embodiment partial proximal ulnar prosthesis according to the present invention;

FIG. 12 is an enlargement of a part circled in FIG. 11;

FIG. 13 is a partial front elevational view of an alternate embodiment partial ulnar or radial prosthesis according to the present invention;

FIG. 14 is a sectional view taken along line 14-14 of FIG. 13;

FIG. 15 is a front elevational view of an alternative embodiment partial ulnar or radial prosthesis according to the present invention;

FIG. 16 is a view similar to that of FIG. 15 of another alternative embodiment partial ulnar or radial prosthesis according to the present invention;

FIG. 17 is a sectional view of yet another preferred embodiment partial distal ulnar prosthesis according to the present invention;

FIG. 18 is a sectional view of yet another preferred embodiment radial prosthesis according to the present invention; and

FIG. 19 is a perspective view of a partial ulnar prosthesis of the present invention connected with an artificial elbow joint.

### DETAILED DESCRIPTION OF THE PREFERRED EMBODIMENTS

The following description of the preferred embodiments is merely exemplary in nature and is in no way intended to limit the invention, its application, or uses.

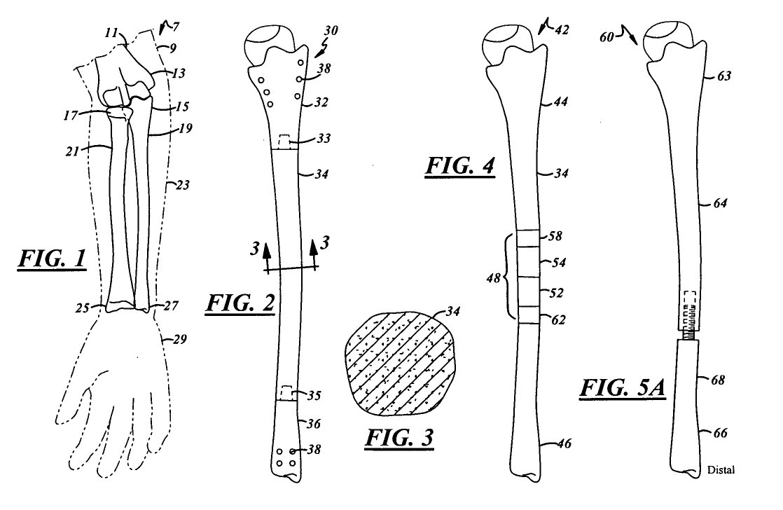

FIG. 1 illustrates a portion of a human arm 7 including a forearm 23. The arm 7 has an upper arm 9 supported by humerous 11. The forearm 23 includes an ulna 19 and a radius 21. The humerous 11 has a distal end 13 pivotally connected with proximal ends 15 and 17 of the ulna 19 and radius 21. Distal ends 25 and 27 of the radius 21 and ulna 19, respectively, are connected with various bones (not shown) of a hand 29. The present invention provides various prosthesis devices for partially or totally replacing the ulna 19 and radius 21.

FIG. 2 illustrates a replacement ulnar prosthesis 30. The ulnar prosthesis 30 has a proximal head 32 connected with an elongated member 34. The elongated member extends a significant length of a patient's ulna. The ulnar prosthesis 30 also has a distal head 36 connected with the elongated member 34. The proximal head 32 and distal head 36 can be integral with the elongated member 34. However, allowing the proximal head 32 and distal head 36 to be separate provides several advantages. The first advantage is that each component of the prosthesis 30 can be customized to a patient's skeletal structure. Additionally, for younger patients, it can be preferable to have an elongated member 34 that is replaceable as the patient physically matures. Additionally, in some applications, it may be desirable that the proximal and distal heads 32, 36 be manufactured from different materials than the elongated member 34. For example, the heads 32, 36 may be manufactured from cobalt chromium, titanium, polyetheretherkeytone (PEEK) and carbon fiber reinforced (CFR) PEEK. It should be noted that the heads 32, 36 may be configured to engage either bone or another type of prosthesis. For example, if the distal humerous 13 (FIG. 1) has been replaced with a prosthetic implant, the proximal head 32 may be configure to engage the distal end of the replacement implant.

The ulna elongated member 34 has a non-uniform cross-sectional shape (FIG. 3) of an actual ulna and can be fabricated from a metal such as titanium alloy Ti-6 Al4V or other suitable alloys or metals. The elongated member 34 can also be fabricated from a polymeric material such as PEEK or CFR-PEEK over long sections to decrease weight. The ulna elongated member 34 can also be fabricated with a metal titanium substrate with a molded polymer encompassing the substrate.

The proximal head 32 is fabricated from a metal such as titanium or other suitable materials as previously described for the elongated member 34. The proximal head 32 has a series of apertures 38 that facilitate tissue attachment. The preferred material for the proximal head 32 is dependent upon its connection with the patient's humerous. If the proximal head 32 is to be connected with a distal humerous, a polymeric proximal head is typically not desirable. The opposite is the case wherein the ulna proximal head 32 engages a replacement for the distal humerous. The proximal head 32 is connected with the elongated member by Morse taper 33. The distal head 36 also connects with the elongated member 34 with a Morse taper 35. The distal head 36 also has tissue attachment apertures 38.

Referring to FIG. 4, an ulnar prosthesis 42 is provided having a proximal head 44 and a distal head 46. The ulnar prosthesis 42 has a segmented elongated member 48. Segments 52, 54, 58 and 62 are included with the elongated member 48 and have a generally circular uniform cross-sectional shape. Segments 52, 54 can be connected with a T-slot and modular locking screw. Additionally, a type of threaded mechanism may be used to interlock the segments. Segments 58 and 62 allow further customization of the total length of the elongated member 48. The proximal and distal heads 44 and 46 respectively can be connected to the elongated member 48 in a manner similar to the connection of the segments 52 and 54.

Referring to FIG. 5A, an ulnar prosthesis 60 is provided. Ulnar prosthesis 60 has a proximal head 63 integrally connected to a proximal portion 64 of the elongated member. A distal head 66 is integrally connected with a distal portion 68 of an elongated member. In a depicted embodiment, the proximal and distal portions 64 and 68 of the elongated member are threadably connected to allow for expansive length adjustment for initial placement and for subsequent adjustment for growing patients. It should be noted that in other embodiments, the threaded connection may be replaced with a combination of a worm and worm gear.

Referring to FIG. 5B, a radial prosthesis 70 is provided having a proximal portion 71 expandable with respect to distal portion 72.

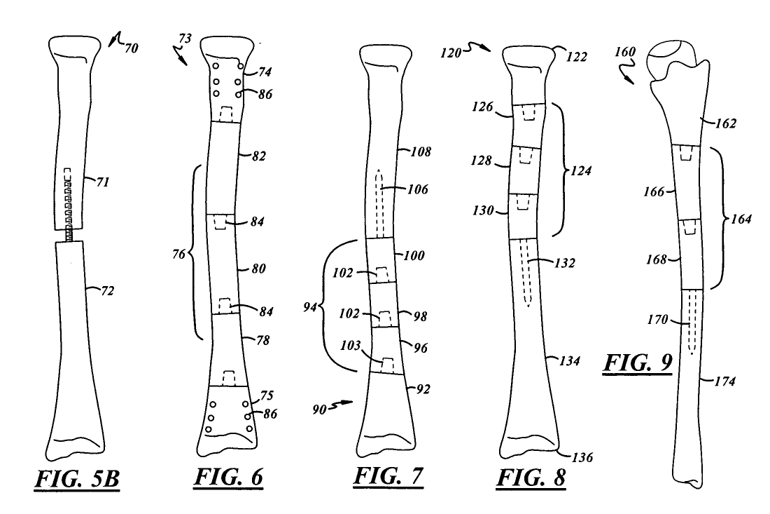

Referring to FIG. 6, a radial prosthesis 73 is provided. Radial prosthesis 73 has a proximal head 74 and a distal head 75. Radial prosthesis 73 has a multiple segmented elongated member 76 having segments 78, 80 and 82 that are connected to one another. In the examples shown, the connective mechanisms are Morse tapers 84. The proximal and distal heads 74 and 75 both have attachment apertures 86. The materials for the radial prosthesis 73 are typically similar or identical to those that have been described for the ulnar prosthesis 30. Additionally, the radial prosthesis 73 can have a non-uniform sectional area of an actual radius.

Referring to FIG. 7, a partial radial prosthesis 90 is provided. Prosthesis 90 has a distal head 92 that is connected by a segmented elongated member 94 having segments 96, 98 and 100 that are connected to one another by Morse tapers 102. Segment 96 of the prosthesis 90 is also joined by a Morse taper 103 with the distal head 92. The segment 100 of the elongated member has connected thereto a stem 106. The stem is connected with a remainder of the patient's radius 108 which by placement within an intra-medullary canal of the radius 108.

Referring to FIG. 8, a radial prosthesis 120 is provided that includes a proximal head 122 joined to a segmented elongated member 124 having segments 126, 128 and 130. Elongated member 124 provides a significant length of a patient's radius and has abutting contact and connection with a remainder of the patient's radius. Segment 130 is connected by a stem 132 in the intra-medullary canal of the remainder of the patient's radius 134.

Referring to FIG. 9, an ulnar prosthesis 160 is provided having a proximal head 162 joined to a segmented elongated member 164 having segments 166 and 168. The elongated member 164 provides a significant length of a patient's ulna and has abutting contact and connection with a remainder distal portion of the patient's ulna 174. Segment 168 is additionally connected with a stem 170 that is placed into the intra-medullary canal of the patient's ulna 174. The materials utilized for the prosthesis 160 can be similar to that as described for prosthesis 90 and 120.

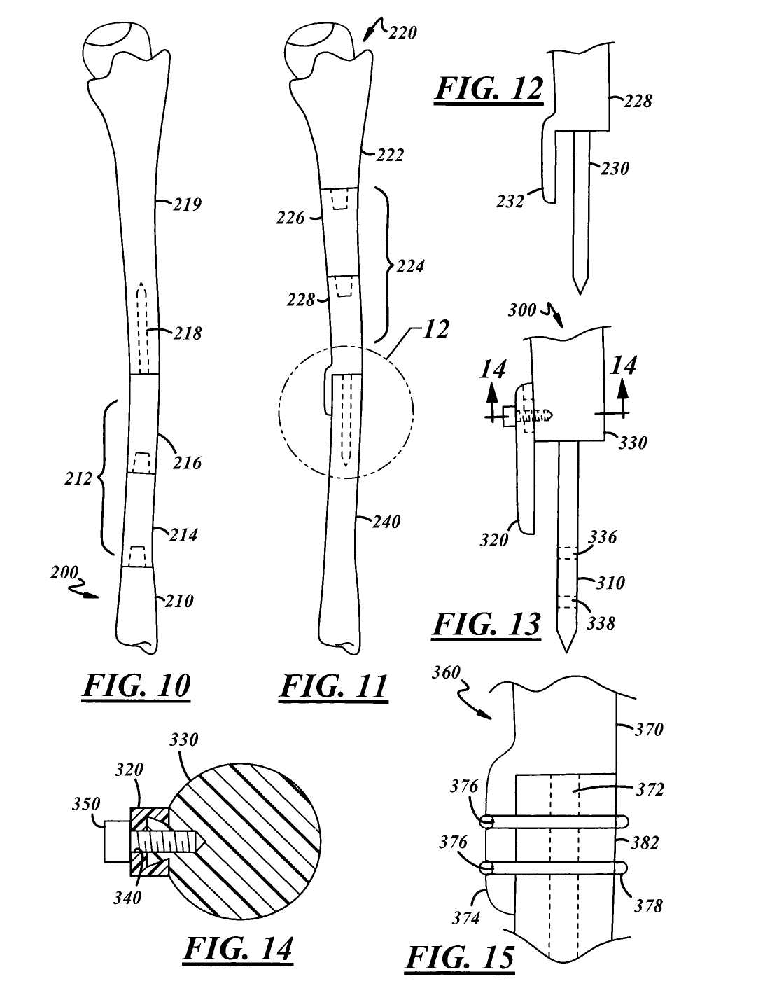

Referring to FIG. 10, an ulnar prosthesis 200 is provided having a distal head 210 joined to a segmented elongated member 212 having segments 214 and 216 wherein the elongated member segment 216 is connected to a stem 218. The stem 218 is connected to the intra-medullary canal of the remainder of the patient's ulna 219. Prosthesis 200 is essentially similar to that afore described prosthesis 160 except that it is for the distal rather than the proximal end of the ulna.

Referring to FIGS. 11 and 12, partial ulna prosthesis 220 is provided having a proximal head 222 that is connected to a segmented elongated member 224 that is inclusive of segments 226 and 228. Segment 228 is connected with a stem 230 and additionally has a flange 232 to enhance its connection with the remainder 240 of the patient's ulna. As shown in FIG. 13, the patient's ulna is the distal portion. However, in other applications (not shown), a segment such as 228 can be connected with the proximal portion of the patient's ulna. In still other applications, a segment of a prosthesis similar to 228 can be configured to combine with the distal or proximal portion of a patient's radius.

Referring to FIGS. 13 and 14, a prosthesis 300 is provided. Prosthesis 300 may be a proximal or distal portion of the ulna or radius. Prosthesis 300 has connected thereto a stem 310 for connection with a remainder of the patient's ulna or radius in the intra-medullary canal as previously described. Additionally, prosthesis 300 has a flange 320 that has a locking mechanism with a main body 330 of the prosthesis 300. In the embodiment shown, the flange 320 provides a dove tail locking mechanism with the main body 330. An elongated slot 340 is provided to allow for passage of a shank of a screw 350 to connect the flange 320 with the stem via the main body 330. The stem 310 has transverse bolt attachment apertures 336, 338 to allow transverse bolts to attach the remainder of a patient's ulna or radius with the prosthesis 300. Slot 340 provides for the axial adjustment of the flange 320 with respect to the body 330.

Referring to FIG. 15, a prosthesis 360 is provided which like afore described prosthesis 300 can be connected with a distal or radial portion of the ulna or radius. Prosthesis 360 has a main body 370 with a connected intra-medullary stem 372 and an integral flange 374. Flange 374 has indentions 376 to facilitate the use of cable(s) 378. The cable(s) can be utilized in a temporary or permanent fashion to further connect the prosthesis 360 with the remainder of the patient's ulna or radius 382.

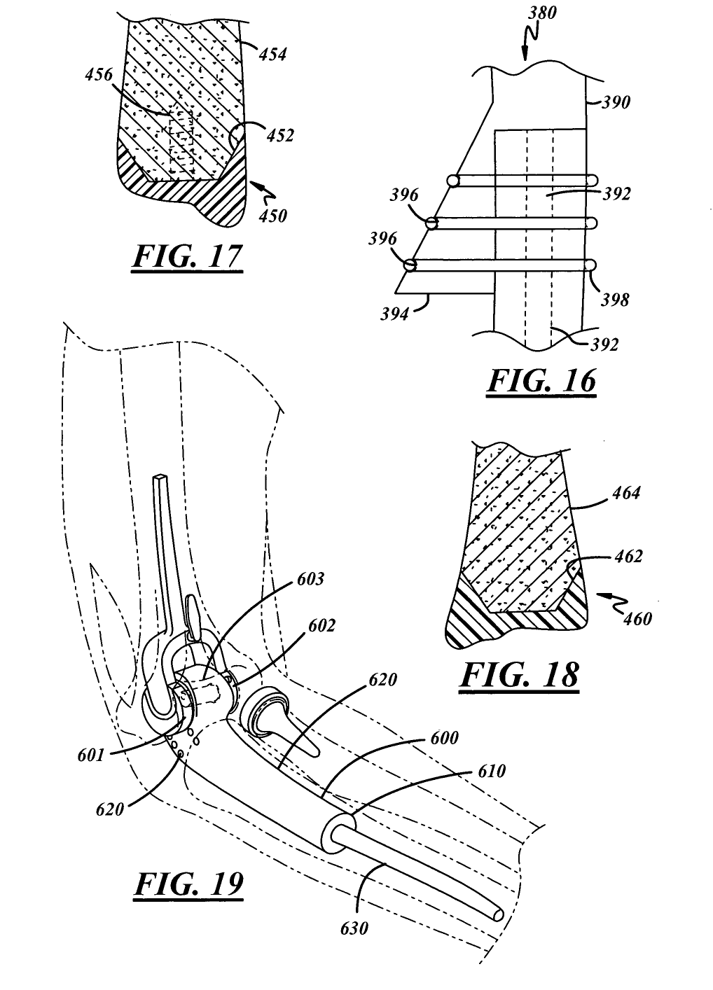

A modification to prosthesis 360 is shown in FIG. 16. Prosthesis 380 (FIG. 16) has a main body 390 with a connected intra-medullary stem 392 with a flared integral flange 394. The flange has indentations 396. The flared flange helps to prevent slippage of the cables 398.

Referring to FIG. 17, an ulnar distal prosthesis 450 is provided with a counter bore 452 for connection with the remainder of the distal end of the patient's ulna 454. Prosthesis 450 in addition to its counter bore 452, may optionally have a stem 456 to connect prosthesis 400 with the remainder of a patient's ulna.

Referring to FIG. 18, a radial distal prosthesis 460 is provided that is connected by a counter bore 462 to the remainder of the patient's distal portion of their radius 464. The distal prosthesis 460 is connected to a prepared portion of the patient's distal radius by locking screws, bone or bone cement.

FIG. 19 provides an ulnar prosthesis 600. The prosthesis 600 has a tubular portion 610 with tissue attachment mechanism 620. This type of ulna prosthesis is best utilized in an elbow type prosthesis that has first and second condylar bearing members 601, 602 with a third bearing member 603 that is operable to articulate with one of the first or second bearing members and which is connected with the ulnar prosthesis 600. The aforementioned elbow prosthesis is further described in U.S. Patent Application Publication US2006/0173546 to Berelsman et al. dated Aug. 3, 2006, the disclosure of which is incorporated by reference herein. Ulnar prosthesis 600 also has at its lower end a sub-stem 630.

The description of the invention is merely exemplary in nature and, thus, variations that do not depart from the gist of the invention are intended to be within the scope of the invention. Such variations are not to be regarded as a departure from the spirit and scope of the invention.

What is claimed is:

1. An ulnar prosthesis comprising:
a head with a counter bore connection with a remainder of a patient's ulna.

2. An ulnar prosthesis as described in claim 1 wherein said head is a distal head.

3. An ulnar prosthesis as described in claim 1 wherein said head is additionally connected to said remainder of the patient's ulna by a penetrating stem.

4. A radial prosthesis comprising:
a head with a counter bore connection with a remainder of a patient's radius.

5. A radial prosthesis as described in claim 4 wherein said head is a distal head.

6. A radial prosthesis as described in claim 4 wherein said head is connected to the remainder of the patient's radius by a penetrating stem.

7. An ulnar prosthesis comprising:
a head;
an elongated member providing a significant length of a patient's ulna being connected with said head, said elongated member having abutting contact and connection with a remainder of said patient's ulna.

8. An ulnar prosthesis as described in claim 7 wherein said head is a proximal head.

9. An ulnar prosthesis as described in claim 7 wherein said head is a distal head.

10. An ulnar prosthesis as described in claim 7 wherein said elongated member is connected to the remainder of the patient's ulna by a penetrating stem.

11. An ulnar prosthesis as described in claim 10 wherein said stem has a transverse bolt attachment aperture.

12. An ulnar prosthesis as described in claim 7 wherein said elongated member is connected to said patient's bone by a flange.

13. An ulnar prosthesis as described in claim 12 wherein said flange has a locking mechanism with respect to said stem.

14. An ulnar prosthesis as described in claim 7 wherein said elongated member is connected to remainder of said patient's ulnar by a penetrating stem and a flange.

15. An ulnar prosthesis as described in claim 14 wherein said flange had indentations for cables.

16. An ulnar prosthesis as described in claim 15 wherein said flange is flared.

17. A radial prosthesis comprising:
a head; and
an elongated member providing a significant length of a patient's radius being connected with said head, said elongated member having abutting contact and connection with a remainder of said patient's radius.

18. A radial prosthesis as described in claim 17 wherein said head is a proximal head.

19. A radial prosthesis as described in claim 17 wherein said head is a distal head.

20. A radial prosthesis as described in claim 17 wherein said elongated member is connected to said patient's radius by a penetrating stem.

21. A radial prosthesis as described in claim 20 wherein said stem has a transverse bolt attachment aperture.

22. A radial prosthesis as described in claim 17 wherein said elongated member is connected to said patient's bone by a flange.

23. A radial prosthesis as described in claim 22 wherein said flange has a locking mechanism with respect to said stem.

24. A radial prosthesis as described in claim 17 wherein said elongated member is connected to remainder of said patient's radius by a penetrating stem and a flange.

25. A radial prosthesis as described in claim 24 wherein said flange has indentions for cables.

26. A radial prosthesis as described in claim 25 wherein said flange is flared.

27. A replacement ulnar prosthesis comprising:
an elongated member;
a proximal head connected with said elongated member; and
a distal head connected with said elongated member wherein at least one head connected with said elongated member having soft tissue attachment mechanisms.

28. An ulnar prosthesis as described in claim 27 wherein said head with soft tissue attachment mechanisms is connected to said elongated member by a Morse taper.

29. An ulnar prosthesis as described in claim 27 wherein said prosthesis is fabricated at least partially from a polymeric material.

30. An ulnar prosthesis as described in claim 27 wherein said prosthesis is fabricated at least partially from a titanium alloy.

31. An ulnar prosthesis as described in claim 27 wherein said elongated member has a non-uniform crossational shape of an actual ulna.

32. An ulnar prosthesis as described in claim 27 wherein said elongated member is segmented.

33. An ulnar prosthesis as described in claim 27 wherein said elongated member is expandable.

34. An ulnar prosthesis as described in claim 27 wherein both said heads have tissue attachment mechanisms.

35. A replacement radial prosthesis comprising:
an elongated member;
a proximal head connected with said elongated member;
a distal head connected with said elongated member; and
wherein at least one head having soft tissue attachment mechanisms.

36. A radial replacement prosthesis as described in claim 35 wherein said head with soft tissue attachment mechanisms is connected to said elongated member by a Morse taper.

37. A radial replacement prosthesis as described in claim 35 wherein said prosthesis is fabricated at least partially from a polymeric material.

38. A radial replacement prosthesis as described in claim 35 wherein said prosthesis is fabricated at least partially from a titanium alloy.

39. A radial replacement prosthesis as described in claim 35 wherein said elongated member has a non-uniform crossational shape of an actual radius.

40. A radial replacement prosthesis as described in claim 35 wherein said elongated member is segmented.

41. A radial replacement prosthesis as described in claim 35 wherein said elongated member is expandable.

42. An ulnar prosthesis for utilization with an elbow prosthesis having first and second condylar bearing members with a third bearing member operable to articulate with at least one of the first and second bearing members, said ulna prosthesis being connected with said third bearing member and said ulnar prosthesis having a tubular body with soft tissue attachment mechanisms and a sub-stem extending therein for insertion in an intra-medullary cavity of a remainder of a patient's ulna.
